(19) United States
(12) Patent Application Publication
(10) Pub. No.: US 2016/0022182 A1
(43) Pub. Date: Jan. 28, 2016

(54) PROBE FOR NON INVASIVE OPTICAL MONITORING

(71) Applicant: OR-NIM MEDICAL LTD., Kfar Saba (IL)

(72) Inventors: Nahum BUDIN, Herzliya (IL); Stanislav LOKSHIN, Rishon LeZion (IL); Uzi MALIMOVKA, Givat Shmuel (IL); Gidon PESACH, Kfar Vitkin (IL)

(21) Appl. No.: 14/775,380

(22) PCT Filed: Mar. 13, 2014
(86) PCT No.: PCT/IL14/50280

§ 371 Date: Sep. 11, 2015

Related U.S. Application Data
(60) Provisional application No. 61/782,641, filed on Mar. 14, 2013.

(51) Int. Cl. A61B 5/1455; A61B 5/00

(52) U.S. Cl. CPC A61B 5/14552; A61B 5/0059; A61B 5/6832; A61B 2562/0257; A61B 2562/0204

(57) ABSTRACT

A probe for use in monitoring one or more parameters of a subject is provided. The probe comprises a monitoring assembly comprising at least one acoustic port for transmitting acoustic radiation into a region of interest in the subject, at least one light output port for transmitting incident light towards the region of interest, and at least one light input port for receiving light returned from the subject. The probe comprises also at least one control mechanism comprising at least one sensing assembly configured for sensing at least one of proximity, attachment and signal quality conditions, and being configured for controlling a condition of coupling between the probe assembly and the subject, enabling to control operation of the monitoring assembly.

### TECHNOLOGICAL FIELD AND BACKGROUND

This invention is generally in the field of medical devices, and relates to a probe device and a monitoring system utilizing such a probe device for carrying out measurements on a subject utilizing ultrasound tagging of light. The invention is particularly useful for monitoring various parameters, e.g. flow velocity and oxygen saturation in blood vessels, capillaries and venules, and oxygen saturation in deep tissues, such as brain, muscle, kidney and other organs.

Various techniques of non invasive monitoring of conditions of a subject have been developed. These techniques include impedance-based measurement techniques, photoacoustic measurements, acoustic measurements (Doppler measurements), and optical measurements (e.g. oxymetry).

Another approach, based on use of ultrasound tagging of light in measurements of various chemical and physiological parameters, has been developed and disclosed for example in WO 06/097910, WO 05/025399, and WO 2009/116029 all assigned to the assignee of the present application. According to the technique of WO 2009/116029, a probe assembly is used for monitoring one or more parameters of a subject, where the probe assembly comprises an acoustic port for transmitting acoustic radiation into a region of interest in the subject, at least one light output port for transmitting incident light towards the region of interest, at least one light input port for receiving light returned from the subject, and a control utility. The latter is configured for controlling at least one condition of a monitoring procedure and enabling the monitoring procedure upon detecting that said at least one condition is satisfied.

### GENERAL DESCRIPTION

The present invention provides a novel probe assembly enabling effective attachment of the probe to the subject (i.e. allowing continuous monitoring of one or more conditions of a subject) and enabling the attachment part of the probe to be disposable.

The present invention takes advantage of the monitoring techniques utilizing the principles of ultrasound tagging of light, for example as disclosed in the above-indicated patent publications WO 05/025399, WO 06/097910, WO 2009/116029 assigned to the assignee of the present application. The probe assembly of the invention is thus configured and operable to irradiate a region of interest with acoustic waves while taking optical measurements on said region of interest.

The inventors have found that with such a probe assembly it is desirable to prevent generation of acoustic and light radiation at conditions other than a measurement session. More specifically, the probe assembly should be configured to enable self-monitoring of its position with respect to a subject, such that upon detecting that the probe assembly is properly attached to the subject's tissue, or that another predetermined condition exists, the probe assembly can be activated to take measurements.

Thus, the present invention, according to its one broad aspect, provides a probe assembly for use in monitoring one or more parameters of a subject. The probe assembly comprises at least one acoustic port for transmitting acoustic radiation into a region of interest in the subject, at least one light output port for transmitting incident light towards the region of interest, at least one light input port for receiving light returned from the subject, and at least one control mechanism.

In some embodiments of the invention, the probe assembly may comprise at least one acoustic port for receiving acoustic radiation from a region of interest in the subject. This may be implemented using the same acoustic port for both transmitting and receiving acoustic radiation, or by using separate acoustic ports for these functions. In the latter case, there are at least two acoustic ports included in the probe assembly, at least one for transmitting and at least one for receiving acoustic radiations.

The at least one control mechanism is configured for controlling a condition of coupling between the acoustic and light output ports and the subject, and controlling the operation of the probe (monitoring procedure) accordingly, e.g. preventing the probe assembly from operation upon identifying that said condition is not satisfied and/or alerting the operator that said condition is not satisfied and/or providing the operator with a quantitative measure of said conditions. Such control mechanism(s) may comprise at least a proximity sensing assembly, an attachment sensing assembly, or a signal quality sensing assembly.

It should be noted that the probe may be shifted between its operative and inoperative states, based on the control mechanism determination, either automatically, or upon user decision.

In some embodiments of the invention, the proximity sensing assembly of the control mechanism comprises a magnetic assembly, which may include a magnetic sensor (detector) and a magnet. The magnetic assembly may operate such that when the magnet is brought proximally to a detection range of the magnetic sensor corresponding to the condition of the desired coupling between the probe assembly and the subject, the magnetic field of the magnet is detected by the magnetic sensor allowing for the acoustic as well as the optical radiation to be safely activated, however, as long as the magnet is outside the detection region of the magnetic sensor the probe's operation is not allowed.

In some embodiments, in addition to or as an alternative to the magnet-based assembly, the control mechanism may include any other proximity detection assembly such as capacitive based assembly, as well as one or more other sensing assemblies, such as optical assembly, ultrasound distance sensing assembly, pressure measurement assembly, or RFID based assembly.

In some embodiments, the attachment sensing assembly of the control mechanism comprises a mechanical or electro-mechanical assembly that utilizes mechanical properties of the probe assembly in order to allow activation of the probe assembly (e.g. activation of the acoustic and/or optical radiation). In some embodiments, the probe assembly is configured as a two-part device, where the two parts are attachable/detachable between them. This enables one part, by which the probe assembly is brought in contact with the subject to be disposable, and enabling the other part to carry the elements of a measurement unit (acoustic and light ports/elements) and be thus a reusable part. It should be understood that in the single- or two-part design of the probe assembly, the probe assembly includes a so-called measurement and contacting/connecting portions, while in the two-part design these portions are attachable/detachable. In the description below these parts/portions are referred to as respectively reusable and disposable parts, but it should be understood that generally both may be disposable or reusable.

In some embodiments, the signal quality sensing assembly of the control mechanism comprises a light emitter (e.g. LED) that may be associated with a logic controller and may be used to wirelessly transmit information from the disposable part to the reusable part or to the control unit. The information may include a serial number for identification of the disposable part, an authentication signal for certifying that the disposable part is a certified one, a counter indicator for the amount of time since the activation of the disposable part, a signal indicating the degree of coupling between the reusable and disposable parts, a signal indicating the degree of coupling between the probe assembly (reusable and/or disposable parts) with the tissue. The information transmitted may also serve for carrying sensor information, e.g. output from optical detectors when positioned on the disposable part.

Thus, according to a first broad aspect of the invention, there is provided a probe for use in monitoring one or more parameters of a subject, the probe comprising: a monitoring assembly which comprises at least one acoustic port for transmitting acoustic radiation into a region of interest in the subject, at least one light output port for transmitting incident light towards the region of interest, at least one light input port for receiving light returned from the subject, and at least one control mechanism comprising at least one sensing assembly configured for sensing at least one of proximity, attachment and signal quality conditions, and being configured for controlling a condition of coupling between the probe assembly and the subject, thus enabling to control operation of the monitoring assembly, e.g. preventing its operation if the at least one condition is not satisfied.

The probe may include a first flexible portion, and a second portion on which the monitoring assembly is mounted, such that pressing the probe against the subject causes a deformation of the flexible portion, thereby reducing a distance between the monitoring assembly and the subject detectable by the proximity and/or attachment sensing assemblies.

The proximity sensing assembly may comprise a magnetic sensing assembly. Preferably, the magnetic sensing assembly comprises a magnet carried by the first flexible portion and a magnetic sensor located at the second portion, the magnetic sensor defines a sensing region in its vicinity and is configured for sensing a magnetic field of the magnet when the magnetic field overlaps with the sensing region. Alternatively or additionally, the proximity sensing assembly may comprise a pressure sensing assembly utilizing techniques known in the art, such as capacitive, resistive and electro-mechanical strain gauges.

The control mechanism may comprise the at least one proximity sensing assembly and the sensing assembly of a different type for controlling the condition of coupling between the probe assembly and the subject. Such at least two different sensing assemblies control the operation of the monitoring assembly, e.g. prevent the operation of the monitoring assembly as long as the coupling condition is not satisfied. In some embodiments, the coupling sensing assembly is a mechanical assembly, and in this case the mechanical assembly may include a switch located on a flexible portion, such that pressing the probe against the subject causes a deformation of the flexible portion thereby activating the switch to allow operation of the monitoring assembly.

According to some embodiments, the first and second portions of the probe are removably attachable to one another.

The flexible portion may be configured as a probe-subject adhesive unit being associated with a probe-subject adhesive media.

According to another broad aspect of the invention, there is provided a probe for use in monitoring one or more parameters of a subject, the probe comprising: a portion carrying a monitoring assembly configured for radiating the subject with acoustic and optical radiation, and a flexible portion by which the probe faces the subject when in operation, and at least one control mechanism comprising at least one proximity and/or attachment sensing assembly located at least partially on the flexible portion, such that pressing the probe against the subject causes a deformation of the flexible portion thereby reducing a distance between the monitoring assembly and the subject detectable by the at least one control mechanism, thereby enabling to control a condition of coupling between the probe and the subject to control operation of the monitoring assembly.

According to yet another broad aspect of the invention, there is provided a probe for use in monitoring one or more parameters of a subject, the probe comprising: a portion that carries a monitoring assembly and is configured for radiating the subject with acoustic and optical radiations, and a flexible portion by which the probe faces the subject when in operation; and at least first and second control mechanisms comprising at least first and second sensing assemblies respectively of same or different first and second types, each is independently operable to control a condition of coupling between the probe and the subject to prevent operation of the monitoring assembly if the at least first and second different sensing assemblies identify that the condition is not satisfied, wherein at least one of the at least first and second sensing assemblies is a proximity sensing assembly located at least partially on the flexible portion, pressing the probe against the subject causes a deformation of the flexible portion thereby reducing a distance between the monitoring assembly and the subject detectable by the at least one proximity sensing assembly.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIG. 1a is a block diagram of an example of a probe assembly according to one aspect of the invention;

FIG. 1b is a block diagram of an example of a probe assembly according to another aspect of the invention;

FIG. 1c is a block diagram of an example of a probe assembly according to yet another aspect of the invention;

FIG. 2 is an isometric upper view of a specific but not limiting example of the probe assembly of the invention;

FIG. 3 illustrates an isometric bottom view of the probe assembly of FIG. 2;

FIG. 4 shows an example of a two-part design of the probe assembly of FIGS. 2 and 3, and illustrates more specifically a flexible part/portion of the probe assembly, which may be a disposable portion; the figure more specifically shows a part of a proximity sensing assembly (magnetic sensing assembly in this example) partially located in the flexible part of the probe assembly;

FIGS. 5 and 6 are cross-section side views of the probe assembly of FIGS. 2-4 showing more specifically two different sensing assemblies of the control mechanism in their activated states (corresponding to inoperative position of the probe assembly, i.e. its monitoring assembly);

FIGS. 7 and 8 are respectively a cross-section side view of a probe assembly according to another embodiment and an isometric view of a flexible part/portion of the probe assembly, which may be a disposable portion, the probe assembly not having a proximity sensing assembly;

FIGS. 9a and 9b show examples of the probe assembly comprising dual sensing mechanical assemblies located inside the probe assembly;

FIG. 10 is a schematic presentation of a two-part design of the probe assembly of the invention comprising several possibilities of controlling mechanisms, specifically a signal quality sensing assembly;

FIG. 11 shows a probe design in which the light source is located inside the probe itself.

### DETAILED DESCRIPTION OF EMBODIMENTS

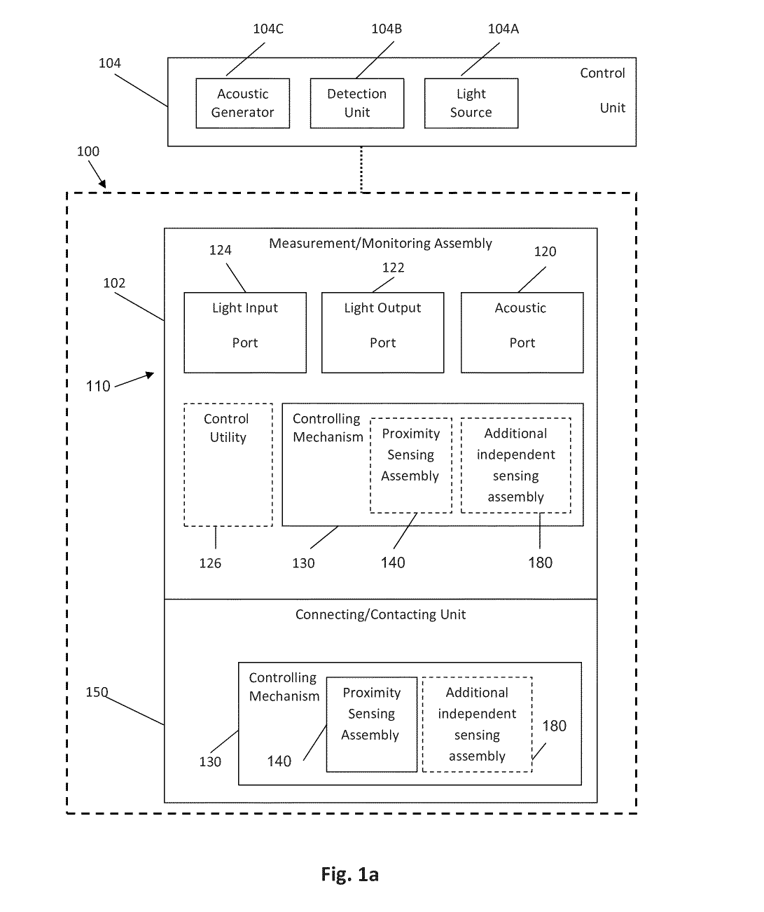

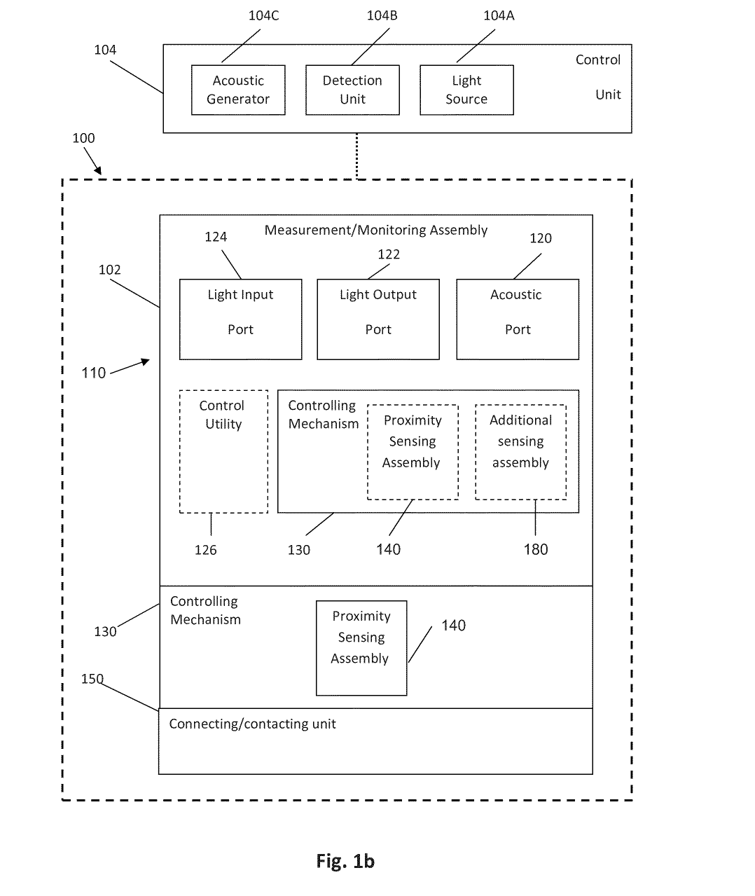

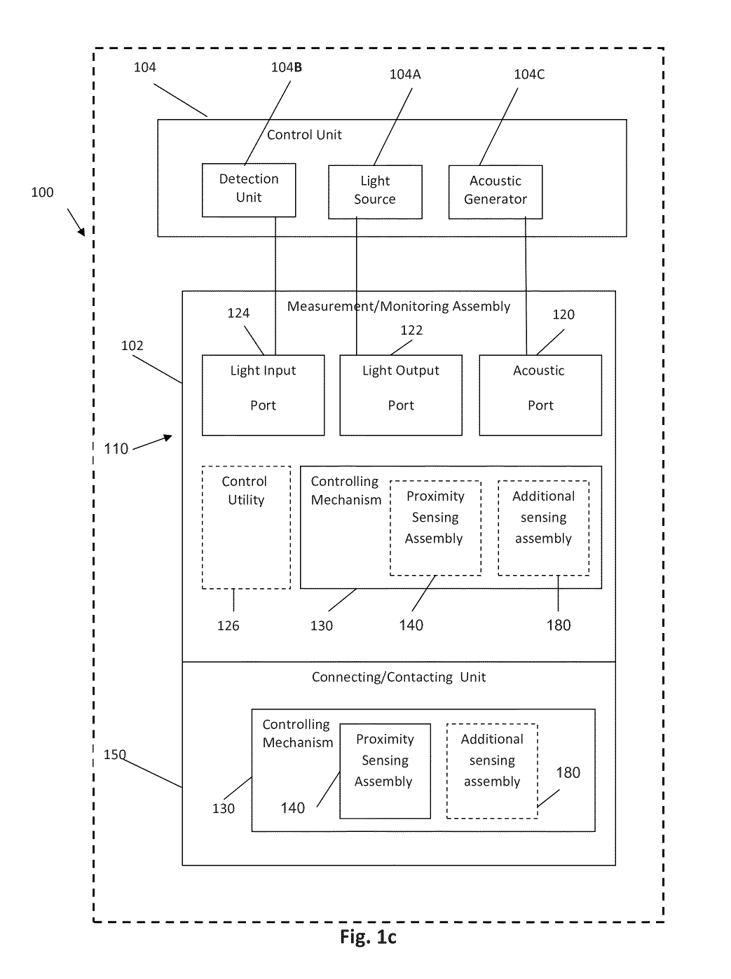

Referring to FIGS. 1a-1c, there is illustrated, by way of a block diagram, three examples of a probe 100 according to the invention for monitoring one or more parameters of a subject. The probe 100 includes a measurement/monitoring unit 102 adapted for carrying out ultrasound tagging based optical measurements, and accordingly includes one or more ports, generally at 120, associated with (i.e. includes or is connectable to) acoustic transducer for transmitting acoustic radiation into a region of interest in the subject, at least one light output port 122 associated with (i.e. includes or is connectable to) a light source for transmitting incident light towards the region of interest, and at least one light input port 124 (i.e. includes or is connectable to) a light detector for receiving light returned from the subject. It should be noted although not specifically shown that the probe may also include additional acoustic transducer for receiving reflected acoustic radiation, or the same acoustic transducer may be configured for transmitting and receiving acoustic radiation. Optionally, the probe may also include, though not shown, indications about its operation mode as well as identification of the illumination condition (e.g. LEDs ON/OFF/Flashing).

The probe assembly 100 may be associated with a control unit 104, which is typically configured as a computing system and logic, and may include among other things a light source unit 104A and/or a detection unit 104B and/or an acoustic generator 104C (e.g. arbitrary waveform generator). Generally, the control unit 104 may be configured as an external unit as shown in FIGS. 1a-1b being connectable to the measurement/monitoring unit 102, or as an internal unit accommodated within the probe assembly 100, as shown in FIG. 1c, or as a hybrid unit (not shown) in which part of the functional units 104A, 104B and 104C are located internally in the probe assembly 100, while the rest are located externally to the probe assembly 100. When configured totally or partially as an external unit, the communication between the probe assembly 100 and the external control unit 104 may be via wires or by means of wireless signal/data transmission (e.g. RF, IR, acoustic).

Preferably, a light guide connecting an external light emitter (e.g. in the control unit) and a light output port at the probe device is a small core fiber (e.g. a single-mode, a 50 μm or a 62.5 μm core fiber). As for a light guide connecting an external light detector (e.g. in the control unit) and a corresponding light input port at the probe device, it has an appropriate cross-sectional dimension of the core in order to satisfy the collection efficiency requirement. For example, a fiber or a fiber bundle, having a core of a diameter equal to or higher than 100 μm can be used. The maximal diameter and numerical aperture of a collecting fiber is determined so that the total path difference between light traveling in different paths in the fiber core is less than the coherence length of the light source. When the light source and/or detector forms an integral part of the probe 100, the optical fibers are eliminated. Electrical wires (or wireless means) are used to connect the probe 100 or the external control unit 104 with a monitor for displaying an image or data related to a diagnosis/monitoring procedure and/or the parameters chosen for carrying out the procedure.

The probe device 100 also includes an internal control utility 126. The control utility 126 may be configured generally similar to that described in the above-indicated patent publication WO 2009/116029 in that it is installed with appropriate electronic utility (coding chip operating with a unique activation code) allowing for identifying whether the probe assembly is a certified one (i.e. authentication procedure); and/or includes a memory unit adapted for recording data indicative of measurements taken on a specific subject during a certain period of time (thus enabling use of this data as a measurement history of the specific subject). Such data may serve as a measurement history of a specific subject, as a measure to record a duration of measurement or an expiration/prevention of use following a specific duration or date. The memory unit may also store data including information about the probe serial-number or any specific technical parameters that are relevant to the probe (e.g. calibration).

The probe device 100 may be configured as a two-part assembly, one part 110 carries the elements of the measurement/monitoring unit 102 and may be reusable, while the other part 150 by which the probe is brought in contact with the subject is configured to provide the desired coupling between the probe and the subject, as well as desired coupling with the reusable part 110, and may be disposable. The two parts 110 and 150 of the probe device are appropriately attachable/detachable to/from one another.

When performing measurements utilizing acoustic radiation, and especially ultrasound tagging of light, a sufficient degree of acoustic coupling between the acoustic port and the subject is needed, as well as certain degree of coupling between the subject and the output light port (to eliminate/reduce eye exposure to the light radiation), so as to actuate and operate measurements upon identifying that desired coupling has been established. According to the present invention, the probe device 100 includes an appropriately designed control mechanism 130, which is configured to satisfy the above two requirements, namely ensure that the measurement/monitoring unit 102 (i.e. reusable part 110) is properly attached to the other, contacting part 150 (disposable), and that said contacting part 150 and the measurement part 110 are attached to the subject under monitoring. To facilitate understanding, the top and bottom parts 110 and 150 of the probe 100 which are functionally different parts, so-called measurement and contacting parts, are referred to hereinbelow as respectively reusable and disposable parts, although it should be understood that generally both may be disposable or reusable.

The control mechanism 130 includes at least a first sensing assembly 140 (such as magnetic and/or optical and/or pressure-based, and/or mechanical and/or electrical and/or resistive etc.), and preferably also includes at least one additional independently operable sensing assembly 180 of the same or different type as the first sensing assembly. Thus, preferably, the control mechanism is a double-sensing mechanism where the two sensing assemblies operate independently, and the measurement procedure is allowed-actuated upon detection by both these assemblies that the proper coupling is achieved.

It should be clear, as can be seen in the FIGS. 1a-1c, that at least part of the control mechanism 130 is located inside the measurement part 110. Another complementary part of the control mechanism may be located outside the measurement part 110, e.g. within the connecting/contacting unit 150 as shown in FIGS. 1a and 1c.

It should be noted that the invention is not limited to the specific configurations shown in the block diagrams of FIGS. 1a-1c, for example another possible embodiment may be a combination of the configurations shown in FIGS. 1b and 1c, i.e. that the control unit 104 is integrated within the probe 100 (as in FIG. 1c) and the control mechanism 130 does not form part of the connecting/contacting unit 150 (as in FIG. 1b).

Referring to FIG. 2, there is schematically illustrated a probe assembly 100 according to an example of the present invention. To facilitate illustration, the same reference numbers are used to identify the common components in all the figures. The probe assembly 100 is constructed from two parts, a reusable unit 110 (carrying/presenting a measurement unit) and a disposable unit 150 (i.e. a contacting part of the probe), which are attached together. The reusable unit 110 includes a housing 112 carrying therein the elements of the measurement/monitoring system. The housing may include a top cover 112A which may be made from rigid plastic. Generally, the top cover 112A may be a single-part structure; however, to facilitate assembling and servicing thereof, it may be composed of multiple parts attachable to one another. In addition, the housing can encapsulate LED for indication of laser emission, or for indication of mode of operation in the case of several probes connected to the same system, each probe may emit light with a different color or signature.

The reusable unit 110 may further include a strain relief member 114 which is configured for securing the connection and bending point of the reusable unit 110 with cables and/or fibers. Optical fibers as well as other cables pass through the member 114 and connect the light input/output ports and the acoustic port, all will be described below, with a proper light source, such as a laser, and an ultrasound transducer respectively. It should be understood that in some embodiments of the invention, the reusable unit 110 includes an ultrasound transducer, while in others it only includes an acoustic port responsible for passing the ultrasound radiation generated by a transducer located outside the reusable unit 110. Further shown in the figure is a strain relief cap 116 that may be used to lock the strain relief member 114. The cap 116 can be manufactured and assembled in various colors to indicate and mark various versions of the reusable unit 110.

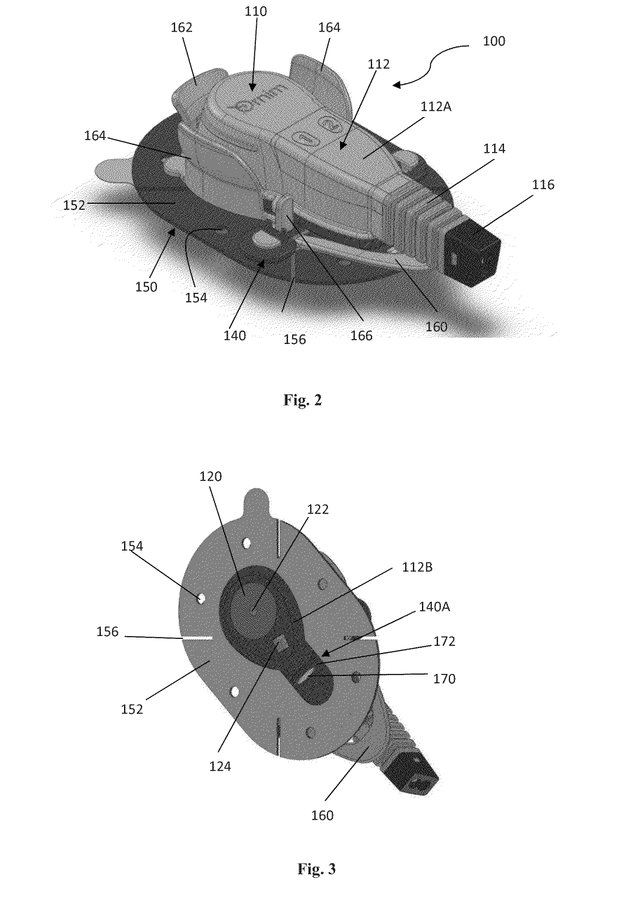

The reusable unit 110 houses various functional elements/units which are not shown in the figure. As described above with reference to FIG. 1, such elements/units include an ultrasound transducer or acoustic port/s for transmitting/receiving acoustic radiation, at least one light output port/source, at least one light input port/detector. Also, the reusable unit carries control mechanisms (e.g. magnetic sensor, mechanical micro-switch), as will be described below with reference to FIGS. 3-5.

The disposable unit 150 includes an attachment pad 152 (such as adhesive pad and/or strap) that assists in attaching the probe to the patient skin. The attachment pad 152 may be relatively wide. The attachment pad 152 may be made of a combination of a bio-compatible adhesive layer and a light blocking fabric layer, both enabling some degree of air ventilation to the patient skin. To support and provide additional ventilation, the pad 152 may include additional through-holes 154. The pad 152 is generally flexible and in order to enhance its flexibility and attachment to the patient's skin matching the curvature thereof, the pad may include additional through-cuts 156 around its circumference. The disposable unit 150 on the one hand serves as a secure support to the reusable unit 110, and, on the other hand serves as a coupler (as an adhesive) of the probe to the patient skin. To this end, the disposable unit 150 includes a support frame 160 which surrounds an opening configured in accordance with the geometry of the reusable unit to be placed therein. The frame 160 is formed with appropriate spaced-apart locking elements 162, 164 and 166 for holding the reusable unit 110 when placed in said opening. More specifically, the support frame 160 has front lock 162, side locks 164 and back supporters 166, which all together operate to position the reusable unit 110 securely and firmly, in place. The front lock 162 prevents a forward motion of the reusable unit 110, and also provides a counter-pressure to the front face of the reusable unit 110 when pushed against the patient tissue. The side locks 164 prevent movements of the reusable unit along the lateral axis (to the sides), and also lock and provide the major counter-pressure to the reusable unit when pushed against the patient tissue. The side locks 164 are preferably designed to enable easy attachment and a single hand removal of the reusable unit. The back supporters 166 prevent backward motion of the reusable part 110 and guide the reusable unit 110 into position when snapped into the disposable unit 150 as will be described further below.

As indicated above, the probe device includes the control mechanism including at least the magnetic sensing assembly 140. In FIG. 2, a part of such mechanism associated with the disposable unit 150 of the probe is partially seen.

Turning to FIG. 3, a rear side surface of the probe assembly 100 of FIG. 2 is schematically illustrated. The figure illustrates the housing 112 of the reusable unit located within/held by the respective opening in the disposable unit 150 and being surrounded by the adhesive pad 152. The bottom side of the reusable unit housing 112 may include a cover 112B made from an elastic material and configured to be received by the opening in the frame of the disposable unit. The bottom cover 112B is also used to protect the reusable unit from the environment (water and dust resistance). The bottom cover 112B holds the acoustic port 120 (or acoustic (ultrasound) transducer as the case may be), the light input port 124 and the light output port 122. As further shown in the figure, a part 140A of the magnetic assembly 140 that is incorporated in the disposable unit 150 includes a holder element 172 holding a magnetic element 170 (e.g. permanent magnet). The configuration is such that the magnet 170 is mounted in the disposable unit such that it is aligned with the other part (not seen here) of the magnetic assembly located in the reusable unit 110.

According to this non-limiting embodiment of the invention, the bottom side of the housing of the reusable unit (e.g. the cover 112B) is configured to enable displacement/deformation of the cover when brought in contact with and pressed against the subject, such that the reusable unit moves towards the subject. To this end, the cover 112B is made of appropriate elastic/deformable material(s) (e.g. elastomeric materials such as rubber or silicone) and possibly also is geometrically designed (e.g. has somewhat curved outer surface) to enable slight movement into and out of the opening. As will be described further below, this configuration is aimed at enabling measurements only upon attaining the desired attachment of the reusable unit with the subject, identifiable by the double-assembly control mechanism. Thus, when the probe device is brought in contact with the subject and a mechanical pressure is applied, the reusable unit and accordingly the acoustic element (port/transducer) and the optical element (input light port(s)) are pushed towards the patient skin, thus providing desired attachment to allow a monitoring procedure to start. When the device is moved away from the subject, the bottom cover 112B returns to its initial shape (e.g. non-deformed state). It should be noted that, in some embodiments of the invention, the cover 112B might be constructed from a rigid/inelastic material being fixed in place, and no movement of the cover would be needed, e.g. if no mechanical micro-switch mechanism (marked 180 in the different figures) is used, or if the mechanical micro-switch mechanism is used in a different configuration being activated by another moving part.

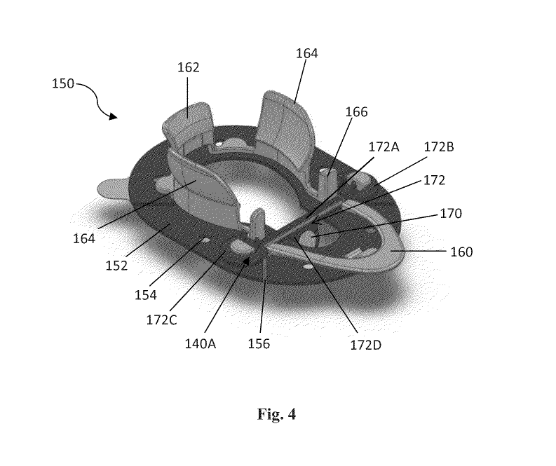

The disposable unit is more specifically illustrated in FIG. 4, showing the magnetic assembly 140A including magnet 170 and the magnet holder 172 which form a part of the control mechanism. As shown, the magnet holder 172 includes a rod-like 172A member which by its opposite ends 172B and 172C is mounted on the opposite side walls of the disposable unit, e.g. mounted on the frame 160, and has a portion 172D carrying the magnet 170. As seen in FIGS. 3 and 4, the configuration is such that the magnet 170 projects from the bottom side of the disposable unit. Also, the configuration is such that the magnet 170 is pivotal with respect to an axis defined by the rod-like holding member. As a result, when the probe device is put in operation and is pressed against the subject, this causes the pivotal movement of the magnet.

According to one possible embodiment, at least the magnet carrying portion 172D of the rod-like member 172A is made from an elastic/deformable material such that when the probe 100 is pressed against a subject, the magnet carrying portion deforms causing rotation of the magnet. Alternatively or additionally, the rod-like element 172A may be rotatable, and thus when the probe is brought to a subject's skin, the magnet carrying portion 172D is pushed causing rotation of the rod 172B which further contributes in the rotation of the magnet.

Thus, the magnetic assembly part 140A in the disposable unit 150 is configured such that a magnet therein is movable towards and away of the reusable unit and thus moves towards and away of a sensing element in the magnetic assembly part installed in the reusable unit. This movement of the magnet towards the reusable unit results in that the magnet 170 becomes located within a sensing region of the magnetic sensing element, which is located inside the reusable unit, thus indicating that the probe is close enough to the subject's skin, being one of the control mechanism conditions.

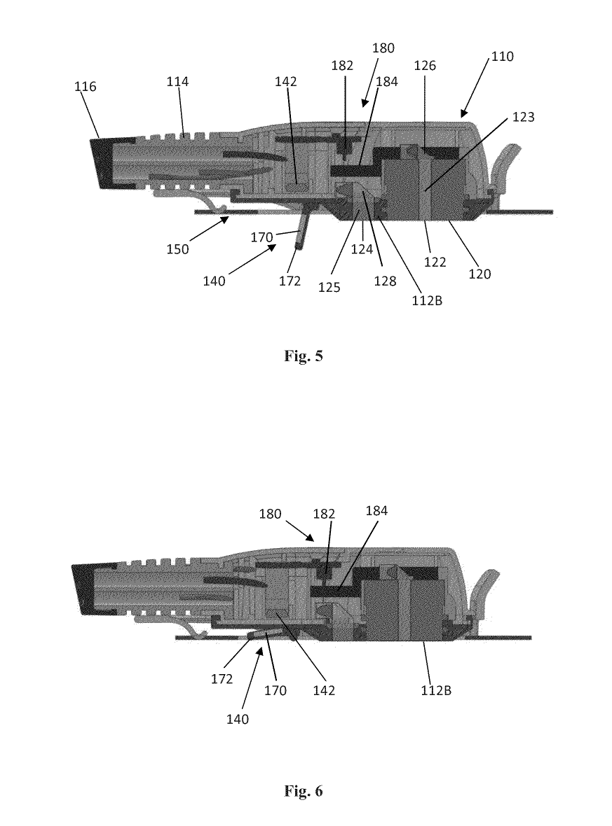

FIGS. 5 and 6 are side cross-section views of the probe assembly 100 according to one possible embodiment of the present invention. Specifically shown in the two figures are two control mechanisms 140 and 180. In this specific not limiting example, the probe assembly is configured to provide double safety check before any radiation can be generated. As described earlier, the control mechanisms allow the safe application of the radiation used during the monitoring procedure, where the activation position of the control mechanisms prevents the probe assembly from operation, i.e. keeps the probe assembly in an inoperative state thereof, while in their deactivated position, the control mechanisms allow the probe assembly to operate. FIG. 5 illustrates the activated state of the two control mechanisms 140 and 180, i.e. the mechanisms prevent the activation of any radiation (optic or acoustic) from the probe 100, and FIG. 6 illustrates the inactivated state of the control mechanisms, i.e. the control mechanisms are neutralized to allow the safe activation of the radiation from the probe 100.

The first control mechanism is the magnetic (generally, proximity-type) sensing assembly 140 which includes the magnet 170 and the elastic magnet holder 172 located in the disposable unit 150, and a magnetic sensing element 142 located in the reusable unit 110 and being at the vicinity of the magnet 170 without physical contact between them. When the probe assembly 100 is not attached properly to the subject, the elastic magnet holder 172 remains in its deactivated position and turns down outside the disposable unit. As a result, the magnetic field of the magnet 170 is not detected by the magnetic sensor 142 and the latter thus does not generate an activating signal. On the other side, when the probe assembly is properly (securely) attached to the subject, the magnet holder 172 changes its orientation upwards into the activated position, as shown in FIG. 6, and consequently the magnetic field of the magnet 170 triggers the magnetic sensor 142, permitting the activation of the radiation source/s. It should be noted that the hysteresis detection range of the magnetic sensor 142 is selected so as to provide an angular range of active-state angles of the magnet 170 which in turn depends on the position of the magnet holder 172, thus enabling the use of the probe over a wide range of patient body curvatures.

It should be noted that, generally, additionally or alternatively, the control mechanism may include any suitable proximity sensing assembly, such as capacitive, optical, ultrasound, mechanical micro-switch, pressure or RFID-based assembly. The construction and operation of such sensing assemblies are known per se and do not form part of the invention, and therefore need not be described in details.

The additional control mechanism may be the mechanical micro switch mechanism 180. This mechanism is located in the reusable unit 110 and includes a micro switch 182 and an elastic lever 184. The latter is located on the bottom cover 112B so as to be aligned with the micro switch 182. This mechanism utilizes the elasticity of the elastomeric bottom cover 112B. As shown in FIG. 5, when the probe assembly 100 is not properly attached to the patient tissue, the elastomeric bottom cover 112B of the reusable unit 110 pops out of the reusable unit's enclosure. In this state, the elastic lever 184 attached to the bottom cover 112B is distanced from and thus not pressing the micro switch 182 located above it, thus the micro switch 182 is not activated. On the other side, when the probe assembly 100 is properly (securely) attached to the patient, as shown in FIG. 6, the elastomeric bottom cover 112B is pushed into the internal cavity of the reusable unit 110, and the elastic lever 184 presses the micro switch 182, thus activating the micro switch 182 to close a respective electric circuit (not shown). It should be noted that the elastic property and the design of the lever 184 provides a larger stroke than the stroke of the micro switch 182, thus extending the available stroke range of the bottom cover 112B and the micro-switch.

FIG. 5 further shows some more details of the reusable unit including the already explained strain relief part 114 and cap 116 alongside with the ultrasound port/transducer 120, the light output port 122 and the light input port 124. It is also shown in this specific but not limiting example that the light output port 122 and the light input port 124 terminate longitudinal rods (light guides) 123 and 125 respectively, along which the transmitted and received light pass. Lenses/prisms 126 and 128 are used to redirect the light to/from the rods from/towards optical fibers (not shown) that mediate between the source/detector and the reusable part 110.

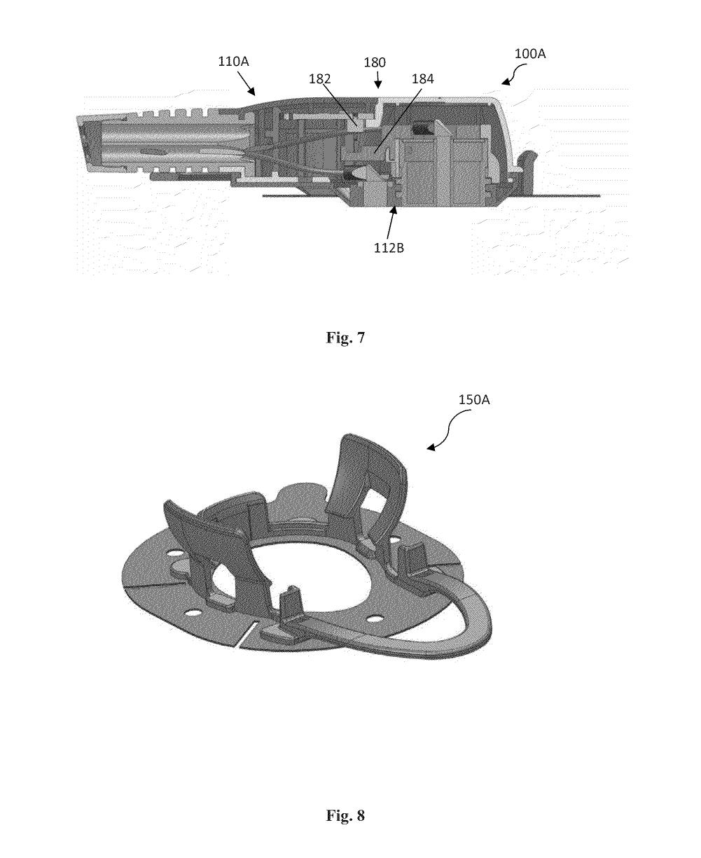

Referring to FIGS. 7 and 8, there is shown another example of a two-part design of a probe 100A according to an embodiment of the present invention. In this particular design, the controlling mechanism/s is/are located inside the reusable part 110A only, and the disposable part 150A provides mechanical support. More specifically, the reusable part 110A, seen in FIG. 7, is built and has the same-structure as the part 110 in FIGS. 5 and 6, except that it does not have a magnetic sensing assembly, i.e. neither the magnetic sensor 142 nor the circuitry associated with it exist. Alternatively, the controlling mechanism of the probe of FIG. 7 includes the mechanical micro-switch mechanism 180 having the same parts and features as explained above with respect to FIGS. 5 and 6, i.e. the mechanism 180 is located in the reusable unit 110A and includes a micro switch 182 and an elastic lever 184. The elastic lever is located on the bottom cover 112B so as to be aligned with the micro switch 182. The mechanism utilizes the elasticity of the elastomeric bottom cover 112B that pushes the micro-switch/es.

The disposable part 150A, shown in FIG. 8, has the same parts and features of the disposable part shown in FIG. 4, except that it neither has the magnet holder 172 nor the magnet 170, as there is no magnetic sensing assembly in this specific configuration. The controlling mechanism 180 of the probe 100A works in the same way as explained above with respect to FIGS. 5 and 6.

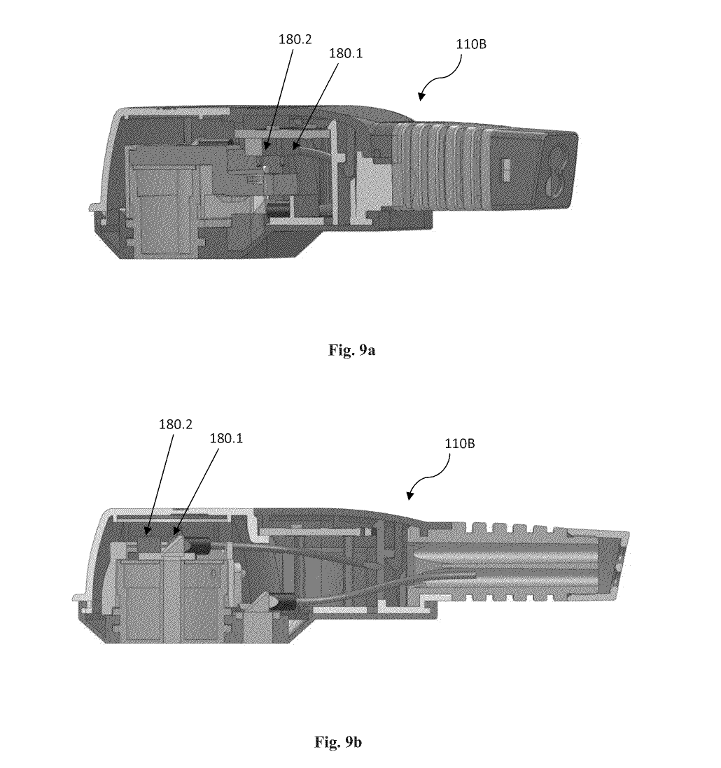

Reference is made to FIGS. 9a and 9b showing other examples of the reusable part 110B of a probe according to embodiments of the invention. The controlling mechanism includes two mechanical micro switches 180.1 and 180.2 that are located inside the reusable part 110B. The reusable part 110B may be used with the disposable part 150A shown in FIG. 8. In FIG. 9b, the micro switches 180.1 and 180.2 are located in direct or indirect contact (e.g. via the transducer structure) with the elastomeric bottom cover 112B, such that when they are pressed against the ceiling of the reusable part 110B a control condition is activated. There is no elastic lever 184 in this design. The fence on the ceiling part of the reusable part 110B limits the movement of the elastomeric bottom cover 112B and keeps the micro-switches and optical lens separated. In these specific examples, only when the two micro switches 180.1 and 180.2 are both activated, the probe is allowed to operate and radiate the acoustic and optical radiations. The dual configuration is advantageous in that it provides safer control of the activation on one side, and allows using cost-effective micro-switches available on the market on the other side. A control logic which may be used in the control unit or elsewhere in the probe assembly, periodically verifies that the two micro-switches provide the same proximity indication, so that any mismatch is indicated as a fault. Accordingly, this would serve as an alert for the user that care should be taken and the device should be repaired. It should be noted that more than two micro switches can be used for ensuring safe operation, with a similar control logic system.

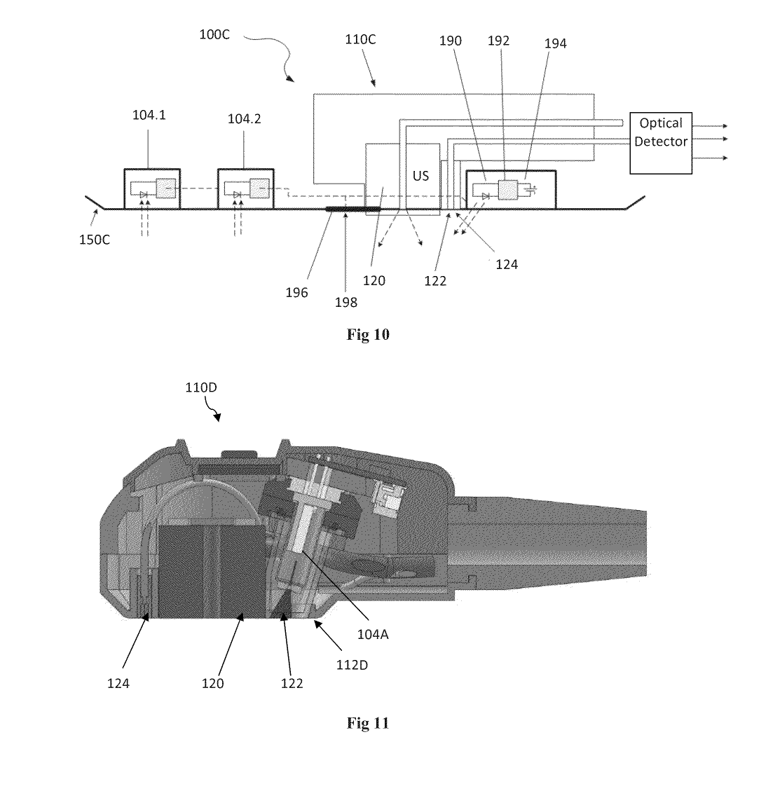

FIG. 10 shows a schematic and functional drawing of more design examples of the design of a single or two-part design of a probe assembly, according to an embodiment of the present invention. In the figure, the probe assembly 100C is configured as a two-part probe including a reusable part 110C and a disposable part 150C. In the reusable part 110C, the probe assembly includes one or more acoustic ports 120, associated with (i.e. includes or is connectable to) acoustic transducer for transmitting/receiving acoustic radiation into/from a region of interest in the subject. The probe assembly also includes at least one light output port 122 associated with (i.e. includes or is connectable to) a light source for transmitting incident light towards the region of interest. Alternatively or in addition to light input port/s 124 integrated in the reusable part and associated with an integral or external light detector, the probe assembly may include one or more light detector/s mounted on the disposable part. In this specific example, two spaced-apart detectors 104.1 and 104.2 are shown and may serve to receive light having one or more of these characteristics: arriving from different regions of the examined subject, having travelled through different paths or depths, being of different wavelengths/frequencies. Either or both detectors can be used for analyzing tagged or untagged light.

In this particular example, as shown in the figure, the controlling mechanism may include one or more of the following components: a light emitting element (e.g. LED) 190 with a logic element (microcontroller) 192 for controlling the light emitter; a power source 194 (e.g. a battery) for the light emitter and/or light detectors 140.1 and 140.2; a pressure sensor 196 and/or an ohmmeter 198 (or resistance meter or electrical current meter). The logic controller is configured to encode or convert signals that are transmitted by the lighting element according to a predetermined code.

The light emitter 190 with the associated logic controller 192 may be used to wirelessly transmit information from the disposable part to the reusable or the control unit (104). The information may include: a serial number for identification of the disposable part, an authentication signal for certifying that the disposable part is a certified one, a counter indicator for the amount of time since the activation of the disposable part (or the logic element), a signal indicating the degree of coupling between the reusable and disposable parts, a signal indicating the degree of coupling between the probe assembly (reusable and/or disposable parts) with the tissue, optical information detected by the light detectors, and more. This degree of coupling may be measured by the pressure sensor 196 or the ohmmeter 198, or any other proximity or attachment sensing assembly used in the invention or is known in the art, encoded by the logic controller to activate the lighting element, and transmitted optically to the control unit located internally or externally to the reusable part. This enables to transmit the information without wires or electrical connections that may unintentionally couple electric currents to the tissue, if not properly isolated. According to the invention, the same light detector may be used for receiving both of the information transmitted by the lighting element 190 and the optical signals that were emitted from the light output port 122. The operation of the lighting element may be synchronized with the operation of the light source included/connected to port 122. This synchronization can be done by firstly sensing by the light detectors on the disposable part that light is not emitted from port 122 and then triggering the operation of the lighting element 190 through the logic controller 192. Alternatively, a communication/synchronization signal can be emitted from light output port 122 to indicate that the light output port 122 will not operate for a specific period of time, allowing operation of the LED 190. It should be noted that the optical link achieved by the lighting element 190 and used for transmitting the information described above, may be substituted by other wireless links and technologies such as RF.

The pressure sensor 196 may be used instead or in addition to any of the previously mentioned proximity and/or attachment sensing assemblies. The pressure sensor 196 may be used for measuring and communicating the amount of pressure that the probe assembly applies on the tissue. The measured pressure is transmitted (through the operation of the lighting element 190 as described above) to the control unit. When the pressure is lower or higher than predetermined minimal or maximal thresholds, the logic controller 192 can display an alert to the user, or cease the operation of the probe assembly completely.

The ohmmeter 198 (or resistance/current meter) measures the resistance between the disposable part 150C and the tissue, or between sensing electrodes mounted on the disposable part, and sends a signal (e.g. through the operation of the lighting element 190) to the control unit. This signal can indicate the coupling between the probe assembly and the tissue, or the amount of gel, or any other coupling substance or media, positioned between the probe assembly and the tissue. When the resistance is lower or higher than predetermined min/max thresholds, the logic controller 192 can display an alert to the user, or cease the operation of the probe system completely.

In the examples above (e.g. FIGS. 5, 6, 7, 9), the light source (104A) is exemplified as being located inside the control unit which is external to the probe (the reusable part). However, as said earlier, the control unit, or part of it, comprising the light source and/or the acoustic transducer and/or the detection unit may form an integral part of the probe assembly, e.g. may be mounted on the reusable part, as long as the conditions for operating the light source inside the probe, such as temperature, are fulfilled. This is shown in FIG. 11, in which the light source 104A is placed inside the reusable part 110D. Also shown in the figure, the light output port 122, the light input port 124 and the acoustic port (or transducer) 120. The light source 104A is located on an elastomeric bottom cover 112D and pressed to the subject together with US transducer and light input port. The reusable housing 110D consists of two parts: a metal part connected to the light source 104A, serves as a heat sink and moves with the light source 104A, and elastomeric bottom cover 112D and plastic part which closes the entire design.

1. A probe for use in monitoring one or more parameters of a subject, the probe comprising:
a monitoring assembly comprising at least one acoustic port for transmitting acoustic radiation into a region of interest in the subject, at least one light output port for transmitting incident light towards the region of interest, and at least one light input port for receiving light returned from the subject, and
at least one control mechanism comprising at least one sensing assembly configured for sensing at least one of proximity attachment and signal quality conditions, and being configured for controlling a condition of coupling between the probe assembly and the subject, enabling to control operation of the monitoring assembly.

2. The probe of claim 1, comprising a first flexible portion, and a second portion on which said monitoring assembly is mounted, pressing the probe assembly against the subject causing a deformation of said flexible portion thereby reducing a distance between the monitoring assembly and the subject detectable by the proximity sensing assembly.

3. The probe of claim 1, wherein the proximity sensing assembly comprises a magnetic sensing assembly.

4. The probe of claim 2, wherein said proximity sensing assembly comprises a magnetic sensing assembly comprising a magnet carried by said first flexible portion and a magnetic sensor located at said second portion, the magnetic sensor defining a sensing region in the vicinity thereof and being configured for sensing a magnetic field of said magnet when said magnetic field overlaps with the sensing region of the magnetic sensor.

5. The probe of claim 1, wherein the controlling mechanism further comprises an additional sensing assembly being of the same or different type as compared to said at least one sensing assembly, and being operable independent from said at least one sensing assembly, said additional sensing assembly being configured for controlling a condition of coupling between the probe and the subject, the at least two independent sensing assemblies enabling to control operation of the monitoring assembly.

6. The probe of claim 5, wherein said additional sensing assembly is a mechanical assembly.

7. The probe of claim 6, comprising a first flexible portion, and a second portion on which said monitoring assembly is mounted, said mechanical assembly comprising a switch located on said flexible portion, pressing the probe assembly against the subject causing a deformation of said flexible portion thereby activating said switch to allow operation of the monitoring assembly.

8. The probe of claim 2, having at least one of the following configurations: (a) said first and second portions are removably attachable to one another; (b) said flexible portion is configured as a probe-subject adhesive unit being associated with a probe-subject adhesive media.

9. (canceled)

10. The probe of claim 2, wherein said at least one sensing assembly configured for sensing the signal quality condition comprises a light emitting element and a logic controller associated therewith.

11. The probe of claim 10, having one of the following configurations: (i) said light emitting element is carried by said flexible portion, (ii) said light emitting element is configured to wirelessly transmit information to one or more other parts of the monitoring assembly or to an external control unit; (iii) said light emitting element is connected to said light output port, the probe comprising a mechanical support comprising a heat sink structure associated with the light emitting element.

12. The probe of claim 11, wherein said light emitting element is configured to wirelessly transmit information to one or more other parts of the monitoring assembly or to an external control unit, said information comprising one or more of the following: a serial number for identification of the flexible portion, an authentication signal for certifying that the flexible portion is a certified one, a counter indicator for the amount of time since the activation of the probe assembly, a signal indicating the degree of coupling between the first and second portions, a signal indicating the degree of coupling between the probe assembly and the subject.

13. (canceled)

14. The probe of claim 1, comprising a light source connected to said light output port, and a mechanical support comprising a heat sink structure associated with the light source.

15. (canceled)

16. A probe for use in monitoring one or more parameters of a subject, the probe comprising:
a portion carrying a monitoring assembly configured for radiating the subject with acoustic and optical radiations, and a flexible portion by which the probe faces the subject when in operation; and
at least one controlling mechanism comprising at least one proximity sensing assembly at least partially located on said flexible portion, pressing the probe assembly against the subject causing a deformation of said flexible portion thereby reducing a distance between the monitoring assembly and the subject detectable by said at least one proximity sensing assembly, thereby enabling to control a condition of coupling between the probe and the subject to thereby control operation of the monitoring assembly.

17. The probe of claim 16, wherein said proximity sensing assembly comprises a magnetic sensing assembly.

18. The probe of claim 17, wherein the magnetic sensing assembly comprises a magnet carried by said flexible portion and a magnetic sensor located at said portion of the monitoring assembly, the magnetic sensor defining a sensing region in the vicinity thereof and being configured for sensing a magnetic field of said magnet when said magnetic field overlaps with the sensing region of the magnetic sensor.

19. The probe of claim 16, wherein the controlling mechanism further comprises an additional sensing assembly being of the same or different type as compared to said at least one proximity sensing assembly and being operable independent from said at least one proximity sensing assembly, said additional sensing assembly being configured for controlling a condition of coupling between the probe and the subject, the at least two independent sensing assemblies enabling to control operation of the monitoring assembly.

20. The probe of claim 19, wherein said additional sensing assembly is a mechanical assembly.

21. The probe of claim 20, wherein said mechanical assembly comprises a switch located on said flexible portion, pressing the probe assembly against the subject causing a deformation of said flexible portion thereby activating said switch to allow operation of the monitoring assembly.

22. The probe of claim 16, wherein said first and second portions are removably attachable to one another.

23. A probe for use in monitoring one or more parameters of a subject, the probe comprising:
a portion carrying a monitoring assembly configured for radiating the subject with acoustic and optical radiations, and a flexible portion by which the probe faces the subject when in operation; and
at least first and second controlling mechanisms comprising at least first and second sensing assemblies respectively of the same or different first and second types, each being independently operable to control a condition of coupling between the probe and the subject to control operation of the monitoring assembly if said at least first and second different sensing assemblies identify that said condition is not satisfied, wherein at least one of said at least first and second sensing assemblies being a proximity sensing assembly at least partially located on said flexible portion, pressing the probe against the subject causing a deformation of said flexible portion thereby reducing a distance between the monitoring assembly and the subject detectable by said at least one proximity sensing assembly.

24. The probe of claim 23, wherein said proximity sensing assembly comprises a magnetic sensing assembly.

25. The probe of claim 24, wherein the magnetic sensing assembly comprises a magnet carried by said flexible portion and a magnetic sensor located at said portion of the monitoring assembly, the magnetic sensor defining a sensing region in the vicinity thereof and being configured for sensing a magnetic field of said magnet when said magnetic field overlaps with the sensing region of the magnetic sensor.

26. The probe of claim 24, wherein the at least one second sensing assembly is a mechanical assembly.

27. The probe of claim 26, wherein said mechanical assembly comprises a switch located on said flexible portion, pressing the probe assembly against the subject causing a deformation of said flexible portion thereby activating said switch to allow operation of the monitoring assembly.

28. The probe of claim 24, wherein said first and second portions are removably attachable to one another.
